(19) United States
(12) Patent Application Publication
(10) Pub. No.: US 2008/0171381 A1
(43) Pub. Date: Jul. 17, 2008

(54) Method and device for the multiplex cells and tissues analysis

(76) Inventors: Hiroyuki Yonekawa, Center Valley, PA (US); Osamu Joji, Center Valley, PA (US)

Correspondence Address: Hiroyuki Yonekawa, 3500 Corporate Pkw., POBox 610, Center Valley, PA, 18034, US

(21) Appl. No.: 11/799,487

(22) Filed: May 2, 2007

Related U.S. Application Data
(60) Provisional application No. 60/880,734, filed on Jan. 17, 2007.

(51) Int. Cl. C12M 3/00

(52) U.S. Cl. 435/287.2

(57) ABSTRACT

This invention is used for the multiplex cells/tissues analysis using 3D cells/tissue culturing functional particles. Functional particle has been used for the variety of biological assays including immunology, biochemistry and molecular biology as well as cell biology.

We combined different particles in same reaction vessel for realizing multiple assays for cells or tissues analysis. These particles are identified by their mark as different color or fluorescence. Then differently identified cells/tissues are analyzed according to their response to the outside signals evenly. We can apply any signals as chemical trigger, light activation, electric triggers, mechanical trigger or temperature shift. In this patent application, we extend to use these functional particles with permeable parts or permeable reaction vessel for the high trough put cells and tissues analysis. If the diffusion speed of these are calibrated correctly, we can measure complex kinetic of cells or tissues response. Also by the nature of 3D matrix assisted cells/tissues culture technique, we can isolate target cells or tissues mechanically by simple manner after culture or stimuli.

### BACKGROUND OF THE INVENTION

Cell or tissue culture is widely used in many biological assays. Then the three dimensional (3D) cell culture technique is getting popular by their high capacity of cell growth and its inert micro environment (ref 1-3). We invented novel protocol for the preparing triple functional micro-particles for 3D cell culture (ref 4).

The 3D cell culture offers a lot of benefits for the biological application as high absorption capacity of target molecules by their high surface/weight ratio and inert micro environment of cells or tissues. Due to the difficulty of preparation and handling, 3D cell culture method need extra procedures, but there is the strong demand for the high through put cells or tissues analysis with 3D culturing technique.

### SUMMARY OF THE INVENTION

This application is the subsequent application of the U.S. Patent Application No. 60/880,734, filed Jan. 17, 2007.

We combined traditional 3D cell culturing technique with functional particles with different signature. We utilize them for the parallel analysis or high through put analysis of cells or tissues. Then, the combination with the permeable membrane or permeable reaction vessel, this assay will generate multiplex analysis for cells or tissue. Such permeable membrane or substrate can work for the kinetic releasing of molecules for the cells or tissues. 3D culturing matrix as soft gel materials can work as inert microenvironment for cells or tissues, so cells or tissue show more accurate response as their original environment. According to their nature of soft gel or reversible nature of sol-gel transition, we can isolate cells, tissues or excreted substances from matrix very easy.

### EMBODIMENT 1

### Preparation of Multiple Cells in Gelatin Particles and Observation

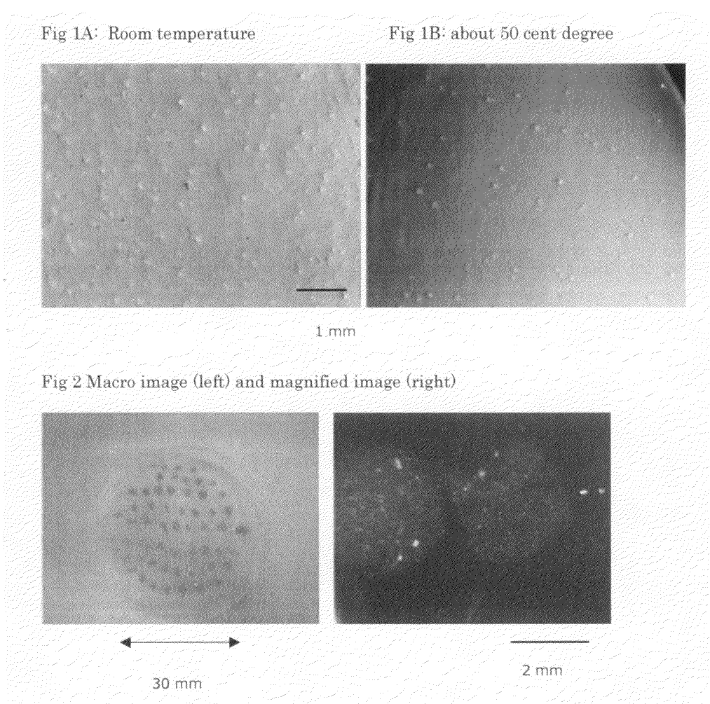

Temperature sensitive gelatin particles are prepared according to the method of ref 5. Gelatin is obtained from Accurate Chemical Scientific Corp, Westbury NY 11590. Arabic gum comes from Senba Touka Kogyou, Japan. Acetic acids come from Heinz PA. Colored dyes come from McCormick, USA. Phosphate buffer, Potassium hydroxide and other chemicals are purchased from Sigma Aldrich, St Louis, Mo. 63178. Heated plate incubator comes from Fisher scientific. Automated mixer was built in-house. Cells come from the mouthwash of human being. We skipped cross-linking step in particle preparation for the reversible transition of sol-gel status. The gelatin particles have reversible transition of sol-gel as seen in (FIGS. 1A & 1B). All images are taken by MIC-D microscope (Olympus America Inc) and Digital camera, CAMEDIA, D-395. Non cross-linked gelatin particles encapsulate cells or tissues in inside very easy and isolate cells or tissues after applying stimuli by increasing temperature.

Preparation of Particles with Different Signatures.

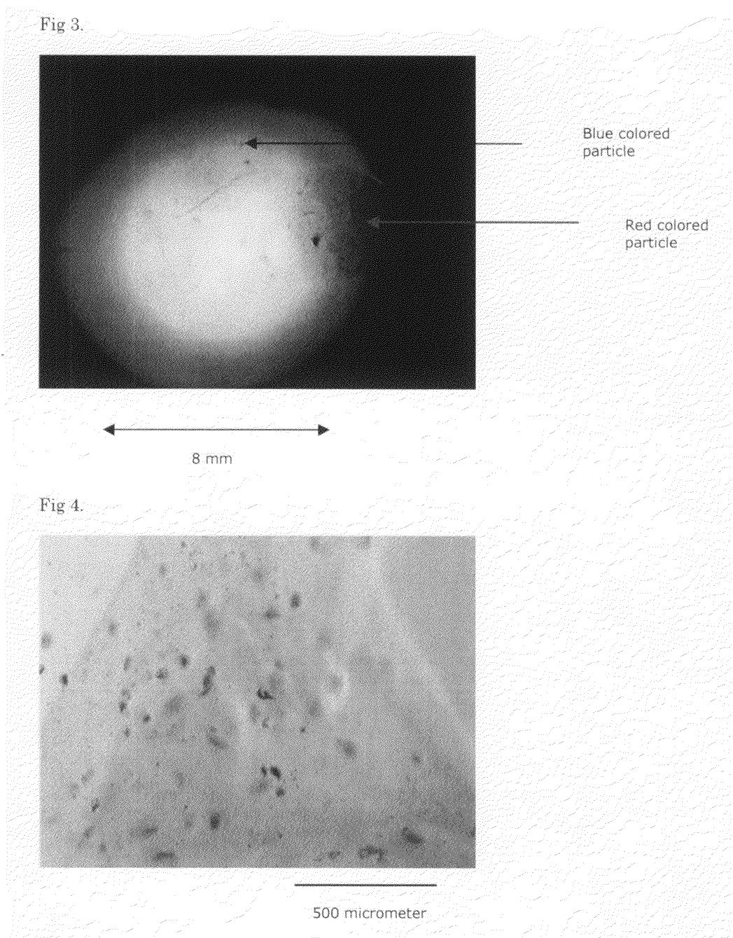

Particles are spiked with colored dye before encapsulating cells or tissue in gelling process. They have different colors and cells as seen in FIGS. 2 A & B. Particle size is controlled by the concentration of gelatin or particle formation process. Two particles are distinguished within single microplate's well easily as seen in FIG. 3. Cells in their particles are also seen easily as FIG. 4.

Gelatin particles are permeable for small molecules, so cells or tissues in the particles can respond to the small.

### EMBODIMENT 2

### Application of Clinical Samples

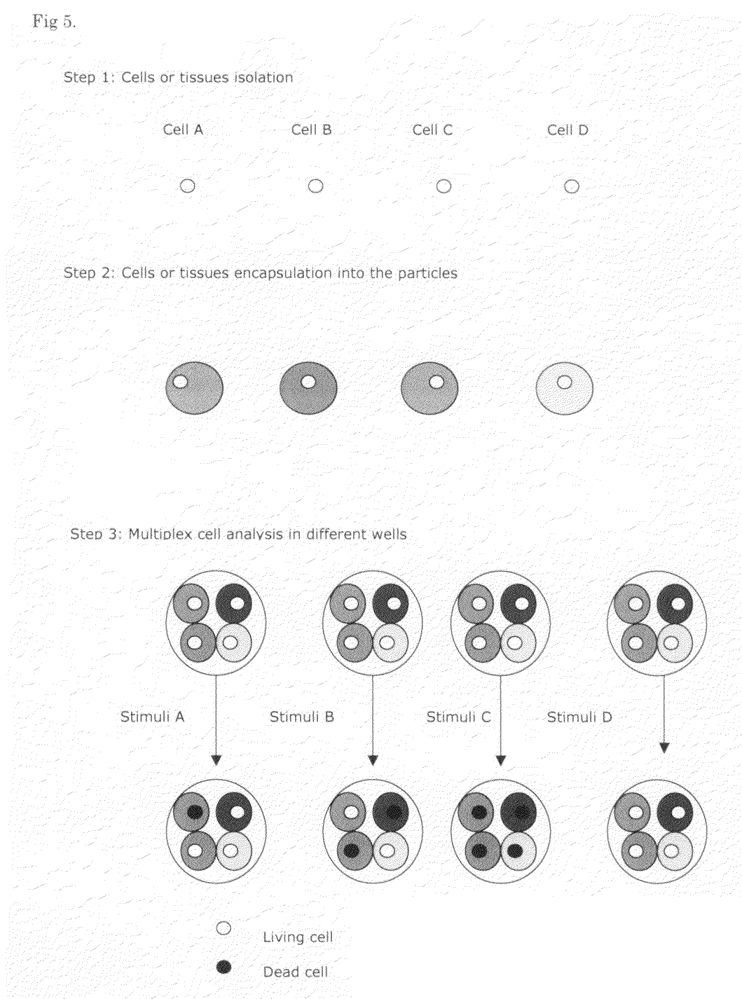

We can apply this method for the clinical investigation of patient samples. Cells or tissues of patient sample are isolated and incorporated into the different particles (FIG. 5). Each particles represent different individuals and response of each cells are analyzed by observation device as responder or non-responder. This method can be used for the drug susceptibility tests. After investigation of cellular response, we can isolate these cells from particle and apply further investigation as cell surface marker or genetic test.

### CROSS REFERENCE TO RELATED APPLICATIONS

Ref 1: U.S. Pat. No. 5,583,037.
Ref 2: U.S. Pat. No. 6,939,709.
Ref 3: InVitrogen Inc, AlgiMatrix 3D Culture System Cat. No. 12684-015
Ref 4: patent Application, U.S. 60/880,734. Filing on Jan. 17, 2007
Ref 5: JPA 2000-275227

### DETAILED DESCRIPTION OF FIGURES

FIGS. 1A & B: Effect of temperature shift on gelatin particles. At the condition of room temperature or about 50 cent degree. These images are taken by MIC-D microscope (Olympus America Inc).

FIG. 2: Cells are encapsulated in different gelatin particles as red or blue. These particles are identified by their color in macro view (left) or micro view (right).

FIG. 3: Two gelatin particles with cells are localized in the microtitier well (standard cell culture ware: diameter of 8 mm).

FIG. 4: Magnification of cells in the gelatin particle.

FIG. 5: Experimental steps of parallel cells analysis method. We can estimate the multiple cells response with fewer experimental steps. Then we can choose suitable combination of stimulus for surviving cells.

### DETAILED DESCRIPTION OF FIGURES

FIG. 1: Effect of Temperature Shift on Two Types of Gelatin Particles.

At the condition of low temperature, two types of particles exist in solution. Then, at higher temperature, above melting temperature of lower melting particles, only particles, which have higher melting temperature, exist in the solution.

FIG. 2: Steps of Cell Encapsulation in Different Colored Gelatin Particles.

Gelatin particles are identified by different colors as indicated by different patterns. Cells are encapsulated into the gelatin particles and identified by their colors. Encapsulation of cells into the gelling matrix is cited in the prior art, U.S. Pat. No. 6,303,151.

FIG. 3: Actual Encapsulated Cells in 3D Gel Matrix.

Human epithelial cells are sampled from human's mouth. Then cells are washed by PBS (Phosphate buffered saline, Sigma-Aldrich (St. Louis, Mo.)) and suspended into the gelatin based 3D matrix. Then cell image is taken by MIC-D microscope (Olympus America Inc,)

FIG. 4: Actual Cells in Two Different Colored 3D Gel Matrices.

Human epithelial cells are sampled from human's mouth. Then cells are washed by PBS (Phosphate buffered saline, Sigma-Aldrich (St. Louis, Mo.)) and suspended into the colored gelatin beads, which have artificially stained by food dyes as red and blue (McCormick, Assorted food colors, St. Paul, Minn.) with the gelatin based 3D matrix. Then cells image are taken by MIC-D microscope (Olympus America Inc,)

FIG. 5: Experimental Steps of Parallel Cells Analysis.

Different cells are encapsulated into the different colored particles by indicated with different patterns. Then, particles are triggered with outside stimulus as chemical, biological or physical manner. Then, we can measure the multiple cellular responses according to the images of cells.

1. Cells or tissues analysis method and device with multiple functioned particles with different signature in same reaction vessel.

2. Multiplex cells or tissues analysis method of claim 1, wherein said cells or tissues are encapsulated in the particles, which have different signatures as color, fluorescence, molecules or magnetic moieties.

3. Multiplex cells or tissues analysis method of claim 1, wherein said cells or tissues are encapsulated in the particles, which are 3D culture matrices as gelatin, fibrin, fiber, cellulose or any cell culturing matrices.

4. Multiplex cells or tissues analysis method of claim 1, wherein said cells or tissues come from the established cell line, stem cells, primary cells or any living organism.

5. Multiplex cells or tissues analysis method of claim 1, wherein said cells or tissues receive outside stimulus as small molecule, light activation, electrical activation, mechanical manipulation, or temperature shift.

6. Multiplex cells or tissues analysis method of claim 1, wherein said cells or tissues are encapsulated and held in separate cubicles by permeable membrane or reaction vessel.

7. Multiplex cells or tissues analysis method of claim 1, wherein said cells, tissues or secreted substances can be isolated and analyzed after culture or applied by outside stimuli.

8. Multiplex cells or tissues analysis method of claim 1, wherein said cells or tissues are encapsulated functional matrix with chemical mediators.

9. Multiplex cells or tissues analysis method of claim 1, wherein said cells or tissues are co-cultured within single particle.

10. Multiplex cells or tissues analysis method of claim 1, wherein said cells or tissues are analyzed by microscope or flow cytometer
